(19) United States
(12) Patent Application Publication
(10) Pub. No.: US 2024/0024648 A1
(43) Pub. Date: Jan. 25, 2024

(54) Sensor Device, Use of a Sensor Device, and Method for Detecting the Properties of a Skin Area

(71) Applicant: LTS Lohmann Therapie-Systeme AG, Andernach (DE)

(72) Inventor: Maral Heidary Dastjerdi, Remagen (DE)

(21) Appl. No.: 18/038,829

(22) PCT Filed: Nov. 23, 2021
(86) PCT No.: PCT/EP2021/082573

§ 371 Date: May 25, 2023

(30) Foreign Application Priority Data
Nov. 26, 2020 (DE) 10 2020 131 377.2

(51) Int. Cl. A61M 37/00

(52) U.S. Cl. CPC A61M 37/0015; A61M 2037/0023; A61M 2037/0061; A61M 2205/3317; A61M 2230/65

(57) ABSTRACT

A sensor device for detecting properties of a skin area, including at least one sensor unit; and a support device connected to the sensor unit. The support device can be connected to the skin area to be detected; the sensor unit includes a transmitter for outputting an electrical and/or magnetic signal to the skin area to be detected, and a receiver for measuring a signal originating from the skin area, which signal results from the signal output by the transmitter. The invention also relates to a use of such a sensor device. In addition, the invention relates to a method for detecting the properties of a skin area.

### CROSS-REFERENCE TO RELATED APPLICATIONS

This application is the United States national phase of International Application No. PCT/EP2021/082573 filed Nov. 23, 2021, and claims priority to German Patent Application No. 10 2020 131 377.2 filed Nov. 26, 2020, the disclosures of which are hereby incorporated by reference in their entireties.

### BACKGROUND OF THE INVENTION

### Field of the Invention

The invention relates to a sensor device for detecting properties of a skin area, the use of such a sensor device, as well as a method for detecting the properties of a skin area.

### Description of Related Art

Beside classic forms of active ingredient administration, such as for example swallowing medicaments or injecting medicine or the like, there are concepts for an application of active ingredients via the skin. In this regard, active ingredient patches and microneedles are of particular importance.

Microneedle arrays, also referred to as microarrays, have a plurality of microneedles that are typically arranged on a support element, such as a patch, a plaster or the like, or connected to a support element. Such microarrays include a high number of microneedles, for example 100 to 600 needles per cm². The needles have a short length, so that upon being pressed into a patient's skin, the needles penetrate the skin only so far that nerves and blood vessels make no contact with needle tips, if possible. The microneedles comprise an active ingredient or a medicament. The corresponding active ingredient may be provided on a surface of the needles or be provided in the needles. It is preferred that the needles are made of a material dissolving in the skin.

For a successful application of a microarray, in particular for a successful medication with microarrays, it is decisive, among other things, that the microarray is applied at the desired skin position, a correct insertion, in particular a correct depth of insertion into the desired skin layer, is performed, a correct/sufficient amount of active ingredient is released, the application is reproducible, in particular independent of the respective skin texture, and/or the active ingredient has been applied exclusively at the desired location and did not subsequently diffuse to the surface, for example.

Presently, there a are no devices and/or methods for evaluating the correct application of microarrays.

### SUMMARY OF THE INVENTION

It is an object of the invention to provide a sensor device for detecting properties of a skin area, a use of such a sensor device, as well as a method for detecting the properties of a skin area, by which the introduction of an agent, in particular an active ingredient, onto and/or into the skin can be detected.

The object is achieved according to the invention by a sensor device, a use of a sensor device, as well a method as described herein.

The sensor device for detecting properties of a skin area according to the invention is particular a sensor device for detecting an active ingredient in a skin area. The sensor device comprises at least one sensor unit. Each sensor unit comprises at least one transmitter. The at least one transmitter is configured to output an electric and/or a magnetic signal to the skin. The sensor unit further comprises at least one receiver. The at least one receiver is configured to measure an electric and/or a magnetic signal from the skin. It is preferred that the receiver is configured to receive, in particular to measure, the signal output by the at least one transmitter. The sensor device further comprises a support device connected to the sensor unit. It is preferred that the support device is formed in one piece, i.e. integrally, with the at least one sensor unit. Preferably, each of the sensor units, in particular all transmitters and all receivers, are formed integrally with the support device. It is possible that the at least one sensor unit is accommodated by the support device, e.g. that the at least one sensor unit is embedded in the support unit. It is also possible that the at least one sensor unit is connected to the support device by form fit or bonding, in particular by gluing. The support device is preferably designed for an arrangement of the sensor device on a skin including the skin area to be detected.

In a preferred embodiment, the sensor device is designed such that the at least one sensor unit is insulated and/or spaced from the skin. In other words, it is preferred that the at least one sensor unit is not in direct contact with the skin. The sensor device is preferably configured such that the at least one sensor unit is in indirect and/or not direct contact with the skin. Here, it is possible that the at least one sensor unit is insulated from the skin by a cover layer of the support device. The cover layer comprises in particular a protective copper lacquer and/or plastic material, e.g. film and/or resin. The at least one sensor unit is preferably embedded in particular in plastic material, e.g. film and/or resin. The sensor unit is preferably embedded in the support device that comprises such materials or is made thereof. In particular, the at least one sensor unit is coated with protective copper layer. Here, the at least one receiver and/or the at least one transmitter is in particular designed to be insulated and/or spaced from the skin. It is preferred that the support device is designed such that an equidistant distance of the transmitter and/or the receiver of the at least one sensor unit to the skin. By the insulation of the at least one sensor unit from the skin, it is in particular achieved in an advantageous manner that signals from the receiver and/or the transmitter are disturbed or distorted by contact with the skin. If the receiver and/or transmitter is configured as a coil, it is in particular achieved in an advantageous manner that, due to the absence of direct contact with the skin, magnetic fields generated by the coil(s) are neither disturbed nor distorted.

It is preferred that the at least one sensor unit is configured such that the transmitter and the receiver are arranged gradiometrically with respect to the skin area to be detected. In particular, the at least one sensor unit is configured to be reflective with respect to the skin area to be detected. The transmitter and the receiver of the at least one sensor unit are preferably arranged parallel to each other and/or parallel to the skin area to be detected. Preferably, the transmitter and the receiver of the at least one sensor unit, as well as preferably also the skin area are arranged vertically offset from each other. It is also possible that the transmitter and the receiver of the at least one senor unit are coaxial with respect to each other. The transmitter of the at least one sensor unit may be within the receiver of that sensor unit, or vice versa. It is preferred that the above relative arrangement of the transmitter and the receiver is realized by an arrangement of the transmitter and the receiver on or in the support device. On the other hand, it is possible that the transmitter and the receiver of the at least one sensor unit are arranged opposite each other on the support device, so that the skin area to be detected is covered by these opposing elements of the sensor unit. Accordingly, a tomographic arrangement of the sensor unit is possible with respect to the skin area to be detected. Thereby, it is possible in an advantageous manner to sense or measure the skin area between the transmitter and the receiver. It is particularly preferred that the sensor device is configured for differential measurement. Thereby, it is achieved in a preferred manner that measurements of the skin area between the transmitter and the receiver of the at least one sensor unit are performed within short periods, in particular within a few seconds or milliseconds. It is possible that at least one coil of the receiver is arranged at a defined angle, e.g. with respect to at least one coil of the transmitter. On the other hand, it is possible to arrange one large transmitter coil and a plurality of small receiver coils in the vicinity of the transmitter coil. This arrangement may be concentric. A sensor array of a plurality of transmitter coils and a plurality of receiver coils is also possible.

In a preferred embodiment, the support device comprises a film. It is preferred that the support device consists of a film. The support film is in particular configured to be adhesive, preferably for sticking on the skin. Preferably, the sensor device is thereby implemented as a film sensor device, also referred to as a film sensor.

It is preferred that the sensor device comprises a fixing device for arranging the sensor unit and/or the support device on the skin area to be detected. Here, it is preferred that the fixing device is designed to be adhesive, i.e. for an adhesive connection with the skin. It is preferred that the fixing device can be separated from the sensor unit and/or the support device.

In a particularly preferred embodiment, the at least one transmitter and/or the at least one receiver comprise a magnet. The magnet preferably is an electric magnet, also referred to as a solenoid. In particular, the at least one transmitter and/or the at least one receiver are made of the magnet. It is preferred that the at least one transmitter comprises a coil, i.e. it preferably consists of the same. If the transmitter comprises at least one coil, it preferably is a transmitter coil. In particular, the at least one receiver comprises a coil, i.e. it preferably consists of the same. If the receiver comprises at least one coil, it preferably is a receiver coil. The transmitter is preferably configured to emit a primary magnetic field, which preferably is a directed magnetic field. In particular, this primary magnetic field causes a Foucault current, also referred to as an eddy current (EC), in the skin. This eddy current in the skin creates a secondary magnetic field which, according to Lenz's law, is preferably opposite to the primary field. It is preferred that the at least one receiver is configured to receive this secondary magnetic field. Here, the received signal is in particular dependent on the dielectric, frequency-dependent electric properties of the respective skin. It is preferred that the receiver is dimensioned and/or designed such that the signal of the transmitter is not detected. This dimensioning and/or design has the advantage that the signal of the transmitter is not too dominant over the signal from the skin and thus, the interesting signal from the skin does not get lost in the noise. It is possible, for example, that the transmitter coil and/or the receiver coil is a coil that can transmit and receive signals, so that it is designed as a transmitter/receiver coil.

It is particularly preferred that the at least one senor unit is configured for eddy current testing, also referred to as EC testing, of the skin area to be detected. It is particularly preferred that the at least one sensor unit comprises, in particular consists of an eddy current testing device, also referred to as an EC testing device. The at least one sensor unit preferably comprises, in particular consists of a scanning coil The sensor device is preferably an EC sensor device.

Preferably, one coil of the receiver of the at least one sensor unit is arranged in particular coaxially inside a coil of the transmitter of this at least one sensor unit. It is particularly preferred that the at least one sensor unit comprises at least, in particular exactly two receiver coils and at least, in particular exactly one transmitter coil, wherein preferably the at least two receiver coils are arranged, preferably coaxially, inside the at least one transmitter coil. In this arrangement comprising at least two receiver coils, it is preferred that the receiver coils are switched opposite each other. In this case, it is preferred not to measure the induction voltage of a receiver coil, but to measure the metrologically more sensitive difference between the receiver coil voltages.

It is preferred that the transmitter of the at least one sensor unit is designed such that it outputs an alternating current, preferably in the β dispersion range. In particular, the transmitter outputs electromagnetic fields of different frequencies and/or frequency ranges. Thus, it is preferred to transmit electromagnetic fields of different frequencies by the transmitter and to preferably receive these by the receiver. Accordingly, it is in particular implemented in an advantageous manner that a frequency spectrum can be received from the tissue, which is characteristic for the area to be examined. This can be compared, for example, to a specific fingerprint. The receiver is preferably configured to receive different frequencies and/or frequency ranges.

Preferably, the sensor device, particularly preferred the at least one sensor unit, are configured such that it is possible to measure the impedance or the alternating current resistance of the skin area to be detected. As such, it is particularly preferred that the sensor device is a bioimpedance spectroscopy device.

In a preferred embodiment, the sensor device comprises at least an evaluation device for receiving and/or evaluating the detected and/or measured data and/or a control device for controlling, in particular for signal injection. It is preferred that the receiver and the transmitter of the at least one sensor unit are connected to the evaluation device in a data- and/or current-transmitting manner via cables and/or conductor paths. The sensor device comprises in particular a terminal for connection to an evaluation device. It is particularly preferred that the terminal is coupleable, so that the connection can be established and terminated physically. The sensor device may e.g. be connectable to the evaluation device via a plug system, e.g. SMA. It is preferred, in particular as an alternative or in addition to a wired connection, that the sensor device, preferably the support device, comprises a communication device for in particular external communication. Preferably, it is a wireless communication device, so that the sensor device can communicate wirelessly in particular with an evaluation device and/or a control device. In particular, the communication device is configured for communication via Bluetooth and/or Wifi and/or radio.

It is preferred that the sensor device, preferably the support device, comprises an energy supply device. The energy supply device comprises in particular a preferably chargeable battery, and is preferably formed by such a battery.

Preferably, the sensor device, in particular the support device, is substantially rectangular, in particular square, or round, in particular circular, or oval. It is preferred that the sensor device, in particular the support device, has a width of 100 mm at most, preferably 10 millimeters at most. As an alternative or in addition, it is preferred that the sensor device, in particular the support device, has a width of 100 mm at most, particularly preferred 10 millimeters at most. On the other hand, the sensor device, in particular the support device, may substantially have a circumference of 100 mm at most, particularly preferred 10 millimeters at most.

In a preferred embodiment, the sensor device comprises a plurality of sensor units, in particular at least four sensor units, the sensor units preferably being arranged in an array.

It is preferred that the sensor device has a marking device for marking the skin area to be detected. It is particularly preferred that the support device comprises the marking device. The marking device is preferably configured such that it marks the skin area to be detected, in particular for a new positioning of the sensor device on the skin area. For example, the sensor device may have a color mark which, in particular after a first arrangement of the sensor device on the skin, is transferred onto the skin and thus marks the arrangement, e.g. “draws” an arrangement circumference of the sensor device on the skin. It is also possible that the marking device marks or identifies the skin area to be detected in an optical manner, e.g. by laser guiding, or in an acoustic manner.

The invention further relates to an application device for introducing an active ingredient into the skin. The application device of the present invention comprises an active ingredient releasing device. The active ingredient releasing device preferably comprises, in particular consists of a microarray or an active ingredient patch. The application device further comprises a sensor device according to one or more of the above described features. It is preferred that the active ingredient releasing device is connected, in particular integrally, to the sensor device.

It is possible in an advantageous manner to perform, in particular simultaneously, an active ingredient release to the skin while at the same time performing measurements using the sensor device. Thus, a direct evaluation of the application can be made in an advantageous manner.

In a preferred embodiment, the application device is configured such that at least one of the receiver and the transmitter is located inside the application region of the microarray and/or the application range of the micro patch. As an alternative or in addition, it is preferred that at least one of the receiver or the transmitter is located outside the application range of the microarray or the application region of the active ingredient patch. When designing the application device with a microarray, it is possible the receiver, the transmitter and the microneedles of the microarray are arranged side by side and/or alternating, or that the sensor unit and the microneedles are arranged side by side and/or alternating.

It is preferred that the active ingredient releasing device of the application device comprises a microneedle support and/or a microneedle applicator. Here, it is preferred that the sensor device is connected, in particular integrally connected to the microneedle support and/or the microneedle applicator. It is preferred that the microneedle support is designed as in DE 10 2019 200 555.8 A1 or in DE 10 2019 200 561 A1. It is preferred that the microneedle applicator is designed as in DE 10 2019 200 563 A1 or DE 10 2019 200 557 A1 or DE 10 2019 122 948 A1 or DE 10 2020109563 A1.

The invention further comprises a use of a sensor device with one or more of the above described features, as well as of an application device with one or more of the above described features. In use, the sensor or application device is used on a surface to be detected, so as to detect the properties of the surface. In particular, the sensor or application device is used on a skin area in order to detect properties of the skin area, in particular to detect an active ingredient in the skin area.

It is preferred that the sensor or application device is positioned several times, in particular twice, on the surface to be detected, so as to detect the properties. In particular, this positioning is made substantially at the same position and/or in the same orientation. Thus, it is in particular implemented in an advantageous manner that the identical surface region can be sensed at different times and/or states, and thus the different states can be compared to one another.

In addition, the invention relates to a method for detecting the properties of a surface region, in particular a skin area. The method according to the present invention is performed using a sensor device. The sensor device comprises at least one sensor unit having at least one receiver and at least one transmitter. It is preferred that the transmitter and/or the receiver comprises, in particular is formed by a coil. The method according to the present invention comprises the steps described in the following, where it is preferred that the steps are executed in the order presented.

A first step is to arrange the at least one sensor unit on the skin including the skin area to be detected for sensing the skin area to be detected. It is preferred that the at least one sensor unit is arranged on the skin as described above with respect to the sensor device. Preferably, the at least one sensor unit is arranged on the skin in a gradiometric and/or reflective manner. On the other hand, a tomographic arrangement of the at least one sensor unit on the skin is possible.

In a second step of the method, a first electric and/or magnetic signal is injected to and/or into the skin area to be detected via the transmitter of the at least one sensor unit. It is preferred that the first signal includes, in particular consists of a primary magnetic field. It is preferred that, by injecting the first signal, eddy currents are generated in the skin area to be detected. The eddy currents preferably generate a secondary magnetic field in the skin area to be detected.

In a third step, a first resultant signal at the skin area to be detected is detected via the receiver of the at least one sensor unit. Preferably, the first resultant signal comprises, in particular consists of the eddy currents generated by the injected first signal or the secondary magnetic field in the skin area to be detected. It is preferred that the impedance of the skin area to be detected is measured.

In a fourth step, the difference, in particular the phase difference and/or the amplitude difference, between the first injected signal and the first resultant signal is measured. Here, it is preferred that the first measuring result is recorded based on this measurement. In an embodiment, in which the at least one sensor unit comprises at least two receiver coils, it is preferred to measure the difference between the receiver coil voltages of these receiver coils.

In a fifth step, which should preferably be executed, a second electric and/or magnetic signal is injected to and/or into the skin via the transmitter of the at least one sensor unit. The second signal is preferably substantially identical to the first signal. It is preferred that the fifth step is correspondingly executed with one or more features of the above described second step.

A preferred step six then includes the detection of a second resultant signal on the skin via the receiver of the at least one sensor unit. It is preferred that the sixth step is correspondingly executed with one or more features of the above described third step.

In a preferred seventh step, the difference, in particular the phase difference and/or the amplitude difference, between the second injected signal and the second resultant signal, is then measured, preferably for recording a second measuring result.

A preferred eighth step comprises the evaluation of the measurements. Here, it is particularly preferred that the evaluation comprises a comparison of the detected measuring results, in particular the first and second measuring results. In a preferred embodiment, any number of further signals can be injected, detected and/or measured, besides the injection, detection and measurement of a first and a second signal.

It is preferred that the execution of the second, third and fourth steps together constitutes a first impedance detection and/or the execution of the fifth, sixth and seventh steps together constitutes a second impedance detection. Preferably, further such impedance detections are possible thereafter.

In a preferred embodiment, the method comprises a further step of removing the at least one sensor unit after the detection of the resultant signal, in particular the first resultant signal. In addition, a step of repositioning the at least one sensor unit prior to the injection of the next, in particular the second electric and/or magnetic signal is preferably executed. Preferably, it is thereby implemented that, first, a positioning of the at least one sensor unit on the skin and a first sensing of the skin area are performed. Thereafter, the at least one sensor unit is preferably removed from the location to be detected. In this manner, an initial state of the skin area to be detected can preferably be detected. Subsequently, it is in particular possible to influence the skin area, e.g. by introducing an active ingredient, in particular by means of a microarray. Thereby it is preferably possible thereafter to again position the at least one sensor unit, in particular at the same position and/or in the same manner and to then perform a new sensing of the skin area so as to change the skin properties after the application, in particular after the introduction of an active ingredient.

It is preferred that the injection of the signals, in particular the first and/or the second signal, is performed for a period of about three seconds. On the other hand, it is preferred that the signal injection occurs for two second, particularly preferred for one second.

It is preferred that an interval lapses between the detection of a resultant signal and a new injection of a further signal. Preferably, an interval lapses between one detection of impedance and a next detection of impedance. If more than two detections, in particular more than two impedance detections, are performed, the intervals in between can be equal, increasing or decreasing. For example, intervals of 0.25 h; 1 h; 6 h; 12 h; 24 h may be provided between a plurality of detections. In this manner, it is in particular possible to advantageously obtain information about a fast or slow release of an active ingredient in the skin area to be detected. This is particularly interesting with microarray patches which dissolve quickly or slowly, e.g. have immediate or sustained release profiles. Thus, it is in particular not only possible to monitor the release, but also to quickly detect side effects such as a sudden burst in active ingredient release, and to take action.

The invention further comprises a method for detecting the introduction of an active ingredient into a skin area. This method comprises the step of executing a method described above. Further, a step of introducing an active ingredient into the skin area is performed. The active ingredient is preferably introduced by means of microneedles or by means of an active ingredient patch. It is preferred that the introduction of the active ingredient is performed after the detection of the resultant signal, in particular after the detection of the first resultant signal. On the other hand, it is also possible to introduce the active ingredient prior to the execution of the above described method for detecting the properties of a surface region.

It is preferred that the above described method for detecting the properties of a surface region, in particular a skin area, and/or the above described method for detecting the introduction of the active ingredient into a skin area are performed using an above described sensor device or an above described application device. Here, it is in particular not necessary that the sensor device comprises a support device when the method is executed. Thus, when the method is executed, the sensor device may comprise one or a plurality of the features of the above described sensor device independent of the support device.

Due to the device/-s and/or method/-s according to the present invention, it is in particular advantageously implemented in the evaluation of microarray applications that: On the one hand, a feedback regarding the application can be obtained, since the impedance of the skin changes after being penetrated by needles; on the other hand, indications about the distribution of active ingredients under the skin and thus a feedback about the efficiency of the microarray delivery, is obtained.

### BRIEF DESCRIPTION OF THE DRAWINGS

In the following, the invention is described in more detail by means of preferred embodiments with reference to the accompanying drawings.

In the Figures:

FIG. 1 is a schematic plan view of a sensor device according to the present invention,

FIG. 2 is a schematic illustration of an embodiment of the sensor device according to the invention in use,

FIGS. 3 and 4 are schematic plan views of further embodiments of a sensor device according to the present invention,

FIG. 5 is a schematic plan view of an embodiment of an application device according to the present invention,

FIGS. 6 and 7 are schematic sectional side views of further embodiments of a sensor device according to the present invention,

FIG. 8 is a schematic sectional side view of a further embodiment of an application device according to the present invention,

FIGS. 9a and 9b are schematic sectional side views of a further embodiment of an application device according to the present invention in two states,

FIGS. 10 and 11 are schematic sectional views of further embodiments of an application device according to the present invention, and

FIG. 12 is a schematic illustration of the method for detecting the introduction of an active ingredient into a skin area according to the present invention with an illustration of the method for detecting the skin properties of a skin area according to the present invention.

### DESCRIPTION OF THE INVENTION

In the Figures, similar or identical components or elements are identified by the same reference numerals or variations thereof (14, 14a and 14b). In particular in the interest of improved clarity, preferably elements already identified are not provided with reference numerals in all Figures. Elements that would not be visible or be covered in the Figures, for example because they are arranged behind other elements, may be illustrated preferably in broken lines for illustration purposes.

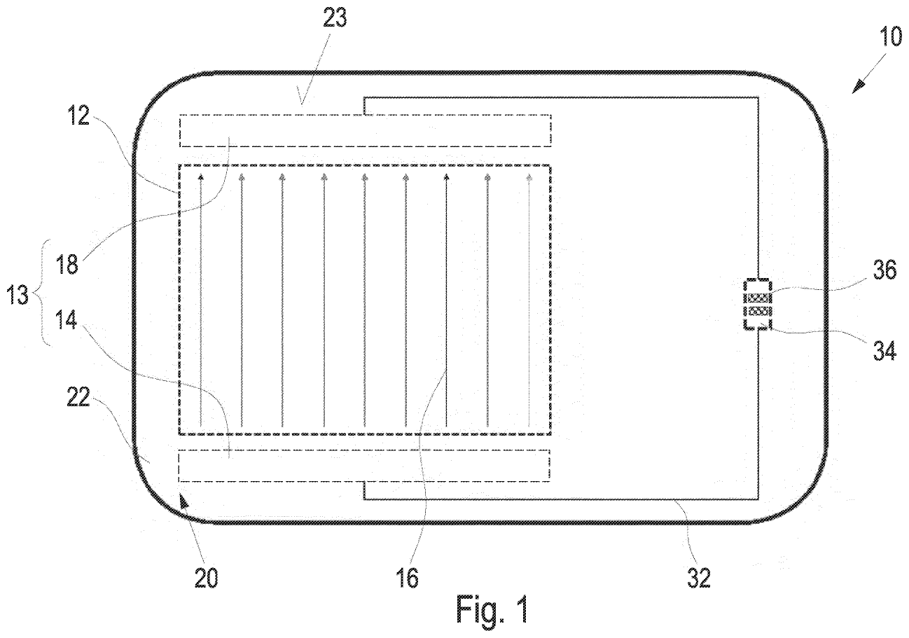

FIG. 1 is a schematic plan view of the lower side of a sensor device 10 according to the invention.

The sensor device 10 comprises a support device 20 which, in the present instance, is designed as a flexible film 22 so that it can be applied, in particular fully, on an e.g. curved location of the body. It is preferred that the lower side of the film 22 illustrated has an adhesive surface 23, which is achieved in particular with an adhesive on the film.

The sensor device 10 has a sensor unit 13 on the lower side of the sensor device 10 illustrated. The sensor unit 13 comprises a transmitter 14 and an opposite receiver 18. In this embodiment, the transmitter 14 shown at the bottom is a transmitter coil 14. The receiver 18 illustrated at the top is a receiver coil 18. In this, as well as in the other embodiments, it is possible that a transmitter coil and/or a receiver coil is a coil that can transmit and receive signals, so that it is designed as a transmitter/receiver coil. The skin area 12 to be detected is located between the coils 14, 18, said skin area being illustrated as an example by the area 12 shown in broken lines. In the embodiment of FIG. 1 showing an opposing arrangement of the receiver 18 and the transmitter 14, a tomographic design of the sensor unit 13 is illustrated.

The transmitter coil 14 outputs a signal, illustrated in an exemplary manner by the arrows 16. The signal output occurs in particular onto the skin area 12 to be detected, on which the sensor device 10 is arranged.

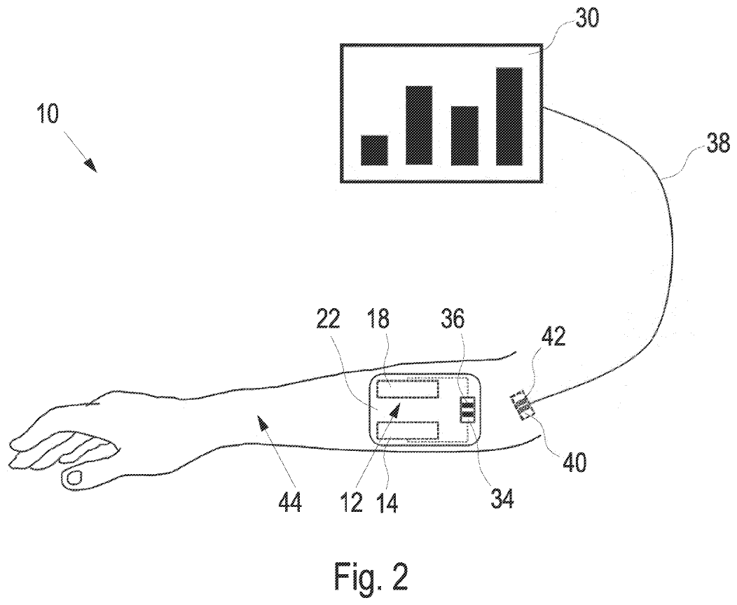

The signal output is in particular an electric and/or magnetic signal. It is particularly preferred that it is a magnetic field. It is preferred that the transmitter coil 14 outputs an alternating current. The receiver coil 18 receives a signal resulting from the output signal 16. It is particular preferred that the impedance is determined by means of the output signal and the received signal. The coils 14, 18 are connected to wires 32, for example for the transmission of data and/or signals and/or current, said wires preferably being conductor paths. The wires 32 allow for a connection to a terminal 34 with contacts 36. It is preferred that the terminal 34 is located on the opposite, front side (not illustrated) of the sensor device 10. It is preferred that the receiver coil 18 and the transmitter coil 14 are located inside within the support device 20 and thus have no direct skin contact. It is particularly preferred that the receiver coil 18 and the transmitter coil 14 are enclosed by the support device. Communication, e.g. with an evaluation device (see, for example, FIG. 2), is possible via the preferably external terminal 34. As an alternative or in addition, it is possible that the sensor device 10, in particular the support device 20 comprises a wireless communication device (not illustrated), for example, for wireless communication with an evaluation device. Preferably, the sensor device 10, in particular the support device 20, may comprise a battery, preferably a rechargeable battery. In particular, the battery is an accumulator.

FIG. 2 schematically illustrates the application of a sensor device 10 according to the invention on a skin area 12, illustrated in the present instance as an area 44 of an arm. It is preferred that the sensor device 10 of FIG. 2 is designed in analogy with the sensor device 10 of FIG. 1.

The film 22 is arranged with its lower side on the skin area 12 of the arm 44 to be detected, in particular by adhesion. A property of the skin area 12 can be determined from output signals and corresponding resultant signals detected at the receiver coil 18, in particular from the impedance determined.

For a current- and/or data-transmitting communication, an evaluation device 30 can be connected via the terminal 34. The connection is preferably made via the contacts 36 of the terminal 34 and the contacts 42 of the corresponding terminal 40 of the evaluation device 30. The evaluation device 30 and the terminal 40 of the evaluation device 30 are connected by a wire 38.

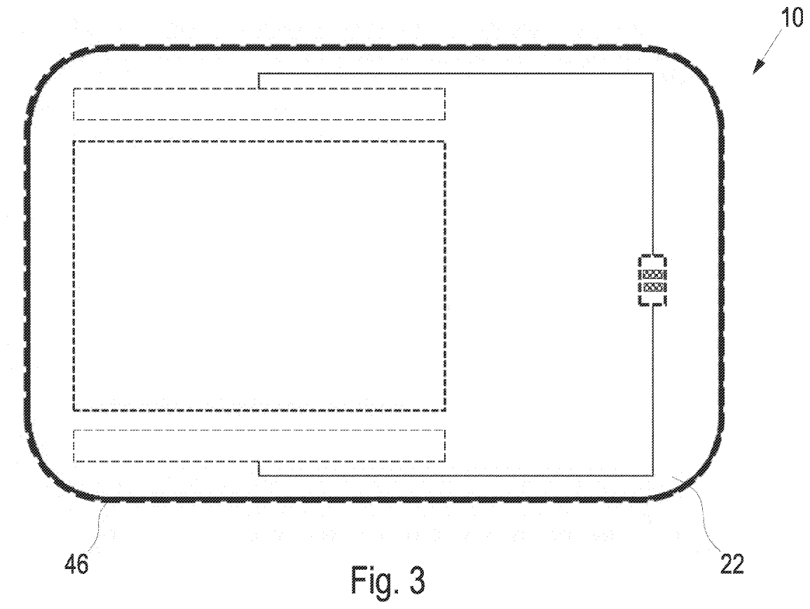

FIG. 3 shows a further embodiment of a sensor device 10 according to the present invention. The sensor device 10 of FIG. 3 substantially corresponds to the embodiment of the sensor device 10 of FIG. 1. The sensor device 10 additionally comprises a marking device 46. As illustrated, the marking device 46 is a color mark provided at the outer edge of the film 22. This color mark is preferably designed such that, when the sensor device 10 is arranged on a skin area, a color mark is transferred onto the skin area. Thus, after removal of the sensor device 10, an identical positioning of the sensor device 10 can be made based on the template-like mark when the sensor device 10 is repositioned on the same skin area.

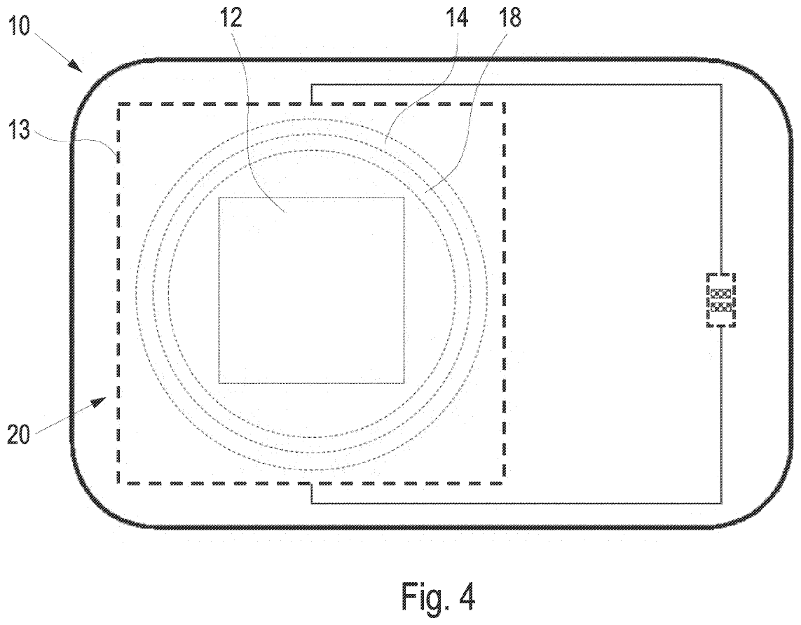

FIG. 4 shows a further embodiment of a sensor device 10 according to the present invention. The sensor device 10 of FIG. 4 is based on the embodiment of the sensor device 10 of FIG. 1. In contrast to the embodiment of FIG. 1, the sensor unit 13 in FIG. 4 is not configured in a tomographic, but in a reflective manner. The transmitter coil 14 and the receiver coil 18 of the sensor unit 13 are arranged coaxially within the support device 20. The transmitter coil 14 preferably emits a primary magnetic field. The primary magnetic field generates eddy currents in the skin area 12 to be detected. The eddy currents preferably generate a secondary magnetic field which can be detected by the receiver coil 18. Instead of the coaxial arrangement of the transmitter 14 and the receiver coil 18, a parallel arrangement of the transmitter coil 14 and the receiver coil 18 is also possible, so that preferably the skin area 12 to be detected is located in parallel below the receiver coil 18 and the receiver coil 18 is located in parallel below the transmitter coil 14.

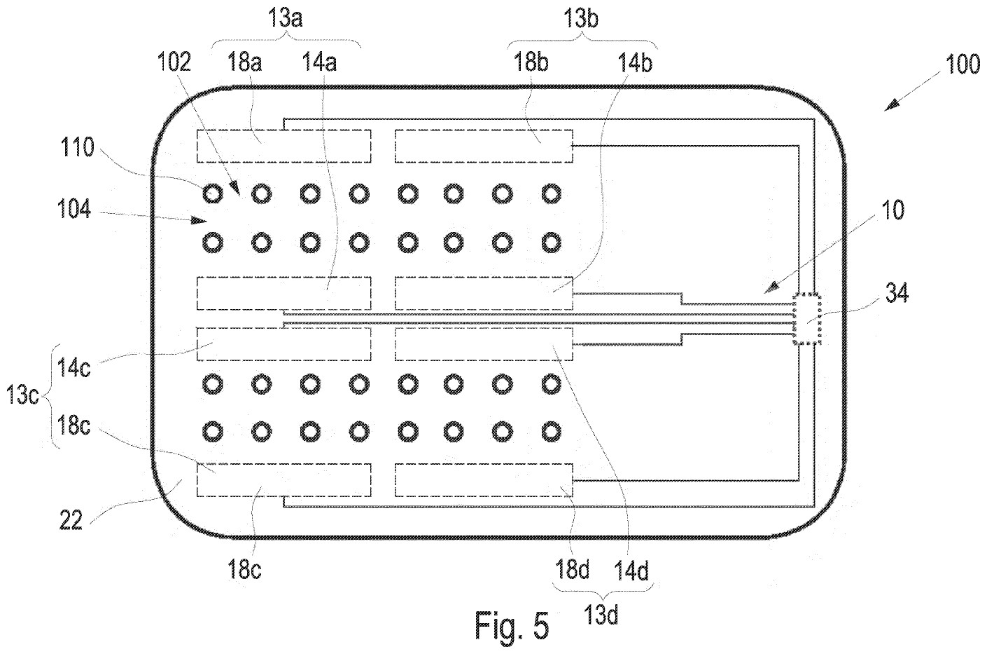

FIG. 5 shows an embodiment of an application device 100 according to the present invention.

On the lower side of the application device 100 illustrated, a sensor device 10 is arranged on the one hand, as well as an active ingredient device, configured as a microarray 104, on the other hand. The microarray 104 comprises a plurality of microneedles 110. Substantially, the film 22 represents the support device for the coils 14a-14d, 18a-18d, as well as a microarray patch for the microarray 104.

Between each of the opposing transmitter and receiver coils 14a and 18a; 14b and 18b; 14c and 18c; 14d and 18d, the property of the skin area located therebetween can be detected, wherein this skin area is influenced in particular by the effect of the microarray 104. This influence may be caused, for example, by the active ingredient introduced and/or the insertion of the microneedles per se. The embodiment of FIG. 5 again is a tomographic sensor device 10. However, it is alternatively possible, for example, that the coils 14a-14d and 18a-18d are each scanning coils and that thus a tomographic arrangement is implemented.

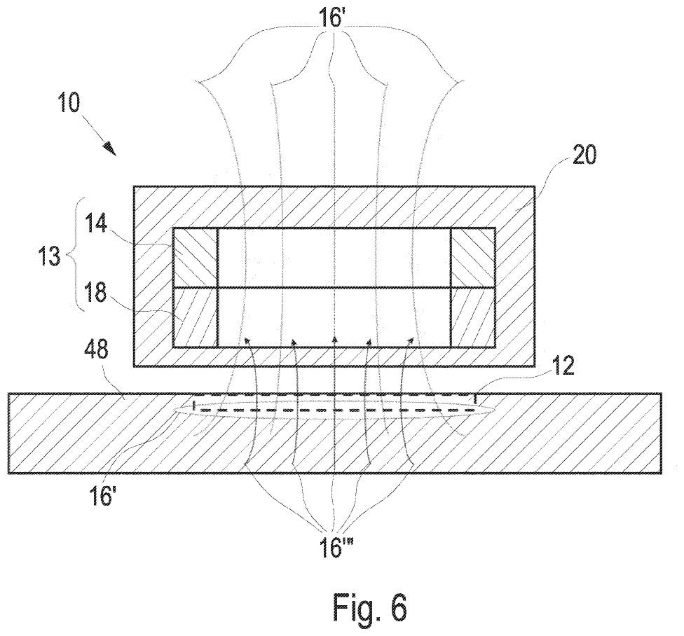

FIG. 6 schematically shows a sectional side view of a further embodiment of a sensor device 10 according to the present invention. A sensor unit 13 comprising a transmitter coil 14 and a receiver coil 18 is arranged within the support device 20 of the sensor device 10. The sensor device, in particular the sensor unit 13, is arranged reflectively with respect to a skin area 12 of the skin 48 of a person, which is to be detected. The transmitter coil 14 emits a primary magnetic field 16′. The primary magnetic field 16′ generates eddy currents 16″ in the skin area 12 to be detected. The eddy currents 16″ preferably generate a secondary magnetic field 16′″ which can be detected by the receiver coil 18.

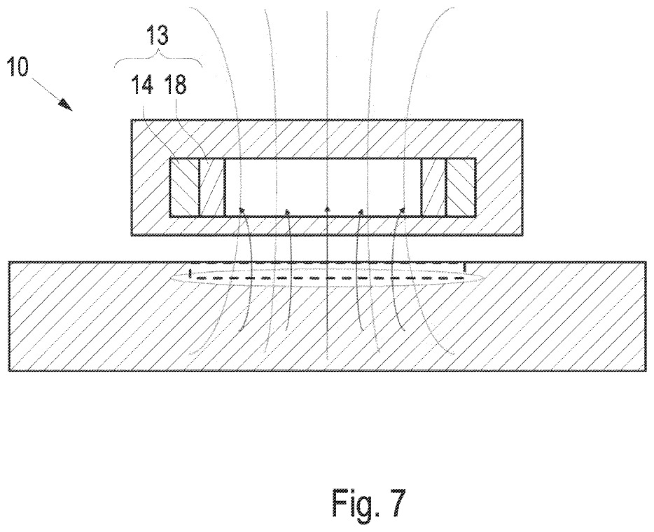

FIG. 7 schematically shows a sectional side view of a further embodiment of a sensor device 10 according to the present invention, which essentially corresponds to the embodiment of FIG. 6. In contrast to the embodiment of FIG. 6, the transmitter coil 14 and the receiver coil 18 are not arranged in parallel one above the other, but coaxially. Here, the receiver coil 18 arranged within the transmitter coil 14.

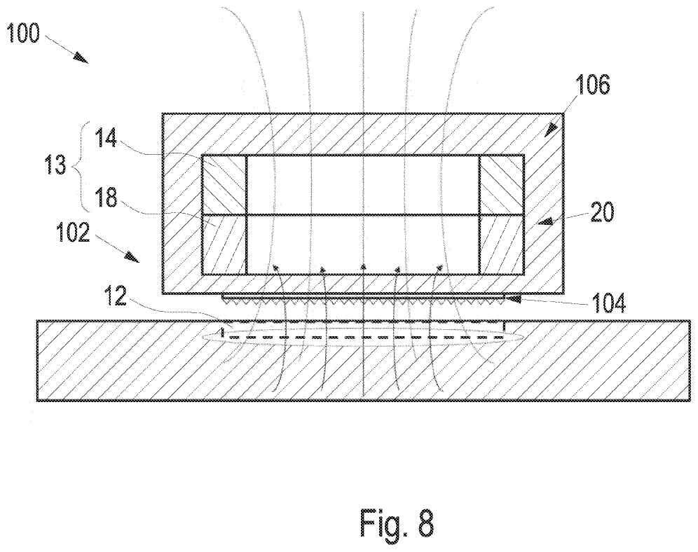

FIG. 8 schematically shows a sectional side view of a further embodiment of an application device 100 according to the present invention with an embodiment of a sensor device 10 according to the present invention. The embodiment of the sensor device 10 substantially corresponds to the embodiment of FIG. 6. The application device 100 comprises an active ingredient release device 102 which comprises a microarray 104, as well as a microarray support 106 supporting the microarray 104. The microarray support 106 is connected, preferably integrally, to the support device 20 of the sensor device 10. In the illustrated, non-applied state of the microarray 104, it is possible, for example, to detect the initial state of the skin area 12 using the sensor device 10. During and/or after an application of the microarray 104 in the skin area 12, it is possible to measure a change by means of a new detection of the skin area 12 using the sensor device 10.

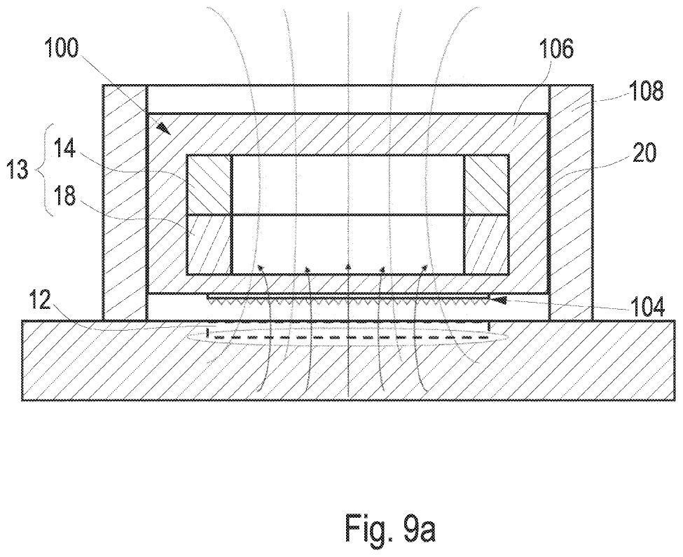

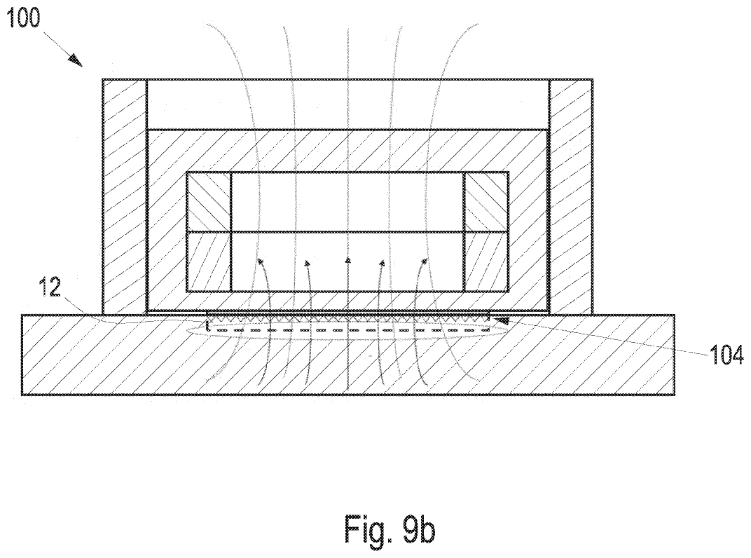

FIG. 9a, 9b schematically show a sectional side view of a further embodiment of an application device 100 according to the present invention with an embodiment of a sensor device 10 according to the present invention. The sensor device essentially corresponds to the sensor device 10 of FIG. 8. The application device 100 is based on the application device 100 of FIG. 8. In addition to the design in FIG. 8, the design in FIG. 9a, 9b comprises a microneedle applicator 108 in which the microarray support 106 is movably arranged for the application of the microarray 104. In FIG. 9a, the microarray support 106 or the microarray 104 is in a non-applied state (at the top). In FIG. 9b, the microarray 104 is applied in the skin area 12 (moved downward). Using the sensor device 10, the skin area 12 can be detected prior to, during and/or after application.

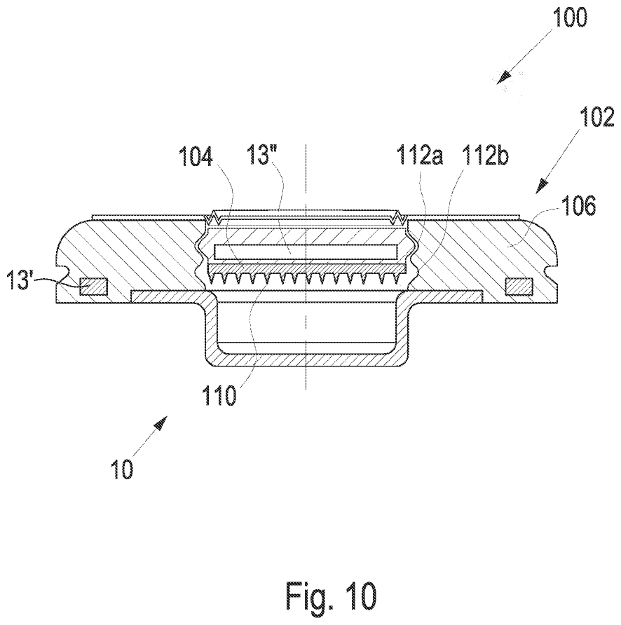

FIG. 10 shows an embodiment of an application device 100 according to the present invention which comprises an active ingredient release device 102 having a microarray 104, as well as a microarray support 106 supporting the microarray 104. Here, the microneedle 106 and/or the microarray 104 are in particular designed as disclosed in DE 10 2019 200 558 A1.

The sensor unit 13 of the sensor device 10 is connected, preferably integrally, to the microneedle carrier 106. The sensor unit 13 is arranged in particular within the microneedle support 106. When the microneedle support 106 is arranged on the skin, the skin area 12, in which the microarray 104 is to be applied, can be detected using the sensor device 10. After an application of the microarray 104 into the skin, i.e. a downward movement of the microarray 104 from the bead 112a to the bead 112b, the skin area also influenced thereby can then be detected (again).

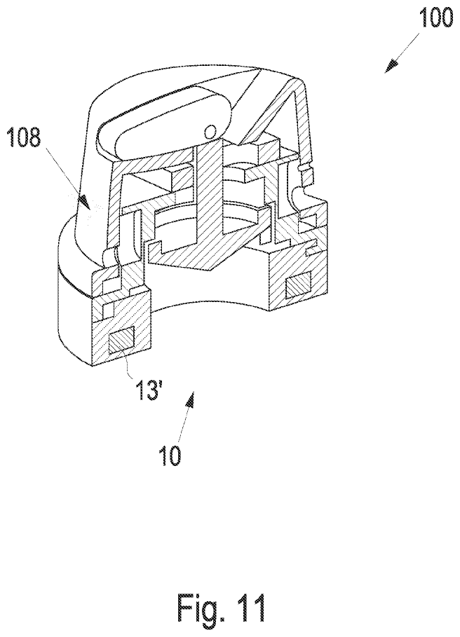

FIG. 11 shows an embodiment of an application device 100 according to the present invention. Here, a sensor device 10 according to the present invention is integrated in a microneedle applicator 108. The microneedle support preferably is the one disclosed in DE 10 2019 200 558 A1. When the microneedle applicator 108 is arranged on the skin, a skin area can be detected via the sensor device 10. In analogy with the embodiment of FIG. 10, this is possible both prior to or after an application.

In all embodiments, in particular in the embodiments of FIGS. 6 to 11, it is possible that the sensor device 10 comprises a plurality of sensor devices 13.

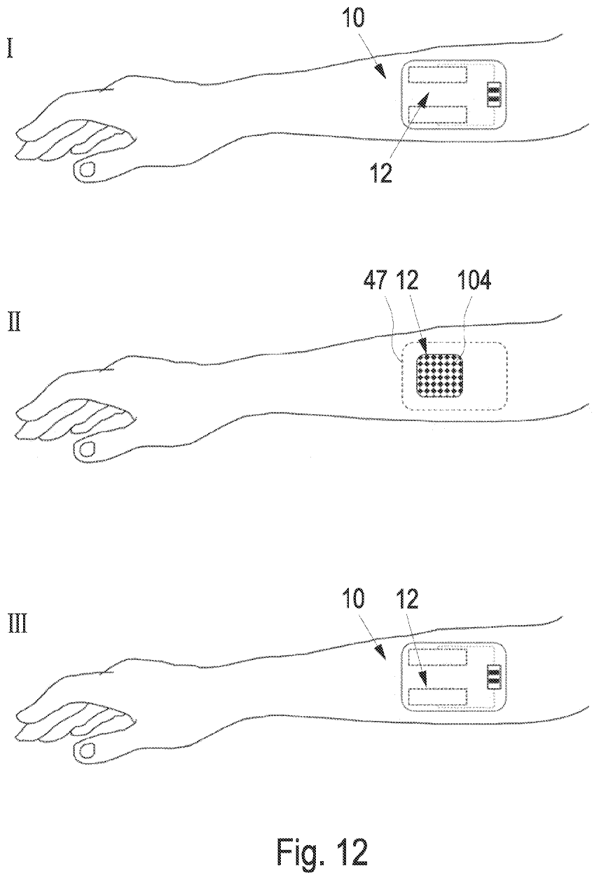

FIG. 12 is a schematic illustration of an embodiment of the method for detecting the properties of a skin area according to the present invention within the framework of an embodiment of a method for detecting the introduction of an active ingredient into a skin area according to the present invention.

I illustrates the arrangement of a sensor device 10 on a skin area 12 to be detected. Thereby, it is preferably possible to detect the initial state of the skin.

II illustrates the introduction of active ingredients into the skin area 12 by means of the microarray 104. For this purpose, the sensor device 10 illustrated in I has been removed from the skin area. Here, it is preferably possible that the first application (I) of the sensor device 10 has left a color mark 47 on the skin area.

III illustrates a repositioning of the sensor device 10 on the skin area 12 to be detected after the introduction of active ingredients (II). Here, it is preferred that the repositioning of the sensor device 10 on the skin area 12 has been made by a template-like positioning of the sensor device 10 on the color mark 47 and that thus a substantially identical positioning was made.

Using the sensor device 10, it is thus possible to detect the property of the skin area 12 to be detected, both prior to an introduction of active ingredients (I) and after an introduction of active ingredients (III). Here, it is advantageously possible to evaluate the active ingredient, introduced in particular by means of the microarray 104.

1. A sensor device for detecting properties of a skin area, in particular for detecting an active ingredient in a skin area, comprising
at least one sensor unit; and
a support device connected to the sensor unit;
wherein the support device can be connected to the skin area to be detected; and
wherein the sensor unit comprises a transmitter for outputting an electrical and/or magnetic signal to the skin area to be detected, and a receiver for measuring a signal originating from the skin area, which signal results from the signal output by the transmitter.

2. The sensor device of claim 1, wherein the support device comprises a preferably adhesive film.

3. The sensor device of claim 1 wherein a preferably adhesive fixing device for arranging the sensor unit and/or the support device the skin area.

4. The sensor device of claim 3, wherein the fixing device comprises a biasing device, comprising in particular at least one spring, for pressing the sensor unit and/or the support device onto the skin area.

5. The sensor device of claim 1, the transmitter and/or the receiver comprise a coil.

6. The sensor device of claim 1, wherein the at least one sensor unit comprises, in particular consists of an eddy current testing device.

7. The sensor device of claim 1, wherein the sensor device, in particular the at least one sensor unit, is configured such that an impedance, preferably of the skin area, can be measured.

8. The sensor device of claim 1, wherein an evaluation device for receiving and evaluating the detected and/or measured data.

9. The sensor device of claim 1, wherein the sensor device, in particular the support device, has a width of max. 100 mm, preferably max. 10 mm, and/or a length of max. 100 mm, preferably max. 10 mm.

10. The sensor device of claim 1, wherein the sensor device comprises a plurality of sensor units the sensor units being arranged in an array.

11. An application device or introducing an active ingredient into the skin, comprising
an active ingredient release device, in particular comprising a microarray or an active ingredient patch, and
a sensor device of claim 1, preferably connected to the active ingredient release device.

12. The application device of claim 11, wherein at least one of the receiver and the transmitter is located within the microarray or the active ingredient patch, and/or that at least one of the receiver and the transmitter is located outside the microarray or the active ingredient patch.

13. The application device of claim 11, wherein the active ingredient release device comprises a microneedle support and/or a microneedle applicator, the sensor device being connected, in particular integrally, to the microneedle support and/or the microneedle applicator.

14. A use of a sensor device claim 1 on a skin area for detecting properties of the skin area, in particular for detecting an active ingredient in the skin area.

15. The use of claim 14, wherein the sensor device is arranged several times, in particular twice, on the skin area to be detected, preferably substantially at an identical position and/or in an identical orientation.

16. A method for detecting the properties of a skin area, with
a sensor device comprising at least one sensor unit having a transmitter and a receiver,
the method comprising the steps of:
arranging the sensor unit on the skin comprising the skin area to detect the skin area,
injecting a first electric and/or magnetic signal at the skin area via the transmitter the at least one sensor unit,
detecting a resultant first signal at the skin via the receiver of the at least one sensor unit, and
measuring the difference between the first injected signal and the first resultant signal, preferably for recording a first measuring result.

17. The method of claim 16, wherein the steps of:
injecting a second electric and/or magnetic signal, which is preferably identical to the first signal, at the skin area via the transmitter of the at least one sensor unit,
detecting a resultant second signal at the skin area via the receiver of the at least one sensor unit,
measuring the difference between the second injected signal and the second resultant signal, preferably for recording a second measuring result, and
evaluating the measurements, in particular the first measuring result and the second measuring result.

18. The method of claim 16, wherein the steps of:
removing the at least one sensor unit after the detection of the resultant signal, in particular the first resultant signal, and
repositioning the at least one sensor unit on the skin to detect the skin area prior to the injection of the next, in particular the second electric and/or magnetic signal.

19. The method of claim 16, wherein an impedance of the skin area is detected when the first resultant signal and/or the second resultant signal are detected.

20. A method for detecting the introduction of active ingredients into a skin area, comprising the steps of:
executing the method of claim 16, and
introducing, preferably by means of microneedles or by means of an active ingredient patch, an active agent into the skin area, preferably after detection of the resultant signal, particularly preferred after detection of the first resultant signal.

21. (canceled)
